(19) United States
(12) Patent Application Publication
(10) Pub. No.: US 2018/0166175 A1
(43) Pub. Date: Jun. 14, 2018

(54) DISCOVERY AND ANALYSIS OF DRUG-RELATED SIDE EFFECTS

(71) Applicant: Georgetown University, Washington, DC (US)

(72) Inventors: Salim Shah, Vienna, VA (US); Howard Federoff, Irvine, CA (US); Ophir Frieder, Chevy Chase, MD (US)

(73) Assignee: Georgetown University, Washington, DC (US)

(21) Appl. No.: 15/576,604

(22) PCT Filed: May 23, 2016
(86) PCT No.: PCT/US16/33715

§ 371 Date: Nov. 22, 2017

Related U.S. Application Data
(60) Provisional application No. 62/165,760, filed on May 22, 2015.

(51) Int. Cl. G16H 70/40; G16H 50/70; G16H 50/20

(52) U.S. Cl. CPC G16H 70/40; G16H 50/20; G16H 50/70

(57) ABSTRACT

Disclosed herein are methods and systems for discovering and analyzing drug related side effects, which are also referred to herein as “off-target responses”. Side effects can be positive/beneficial side effects or negative/undesirable side effects. Further, the positive side effects can be utilize to repurpose a drug while undesirable side effects can be eliminated to make the drug(s) safer. Disclosed methods can utilize any one or more of a variety of data sources and data collection techniques to acquire data that can be utilized to identify side effects related to a particular drug and to determine that causal links between the drug, the patients, and the side effects.

### CROSS-REFERENCE TO RELATED APPLICATIONS

This application claims the benefit of U.S. Provisional Patent Application No. 62/165,760 filed May 22, 2015, which is incorporated by reference herein in its entirety.

### FIELD

This application is related to the discovery and analysis of drug-related side effects using novel data sources and data collection techniques.

### BACKGROUND

Many drugs have been found to cause beneficial effects in patients. However, many drugs also cause undesirable side effects. Some side effects are difficult to detect, and some side effects are difficult to correlate with the use of a particular drug. Moreover, it can be difficult to determine why certain side effects occur is some patients who are taking a particular drug, but those same side effects do not occur in other patients taking the same drug.

Causal links between a particular drug and side effects can be related to inherent characteristics of a drug (e.g., the chemical compound(s) in the drug), inherent characteristics of the patient taking the drug (e.g., genetics, family history, gender, age, current diseases), environmental factors for the patient (e.g., pollution, pollen, weather), behavioral factors for the patient (e.g., occupation, lifestyle, exercise, diet, other drug use), and/or other factors.

Some side effects and their causal links can be determined during pre-clinical drug development and clinical trials and such information is typically available to patients who are taking the drug. However, many side effects and their causal links are not found prior to the drugs being broadly administered to patients, namely, post Food and Drug Administration (FDA) approval, which can have serious consequences. Thus, there is a need in the art for improved methods and systems for detecting and analyzing a more comprehensive set of drug-related side effects using a more comprehensive range of data sources and data collection techniques that take into account the broad range of possible causal links between a particular drug and its side effects.

### SUMMARY

Disclosed herein are methods and systems for discovering side effects of particular drugs using traditional and non-traditional data sources and data collection techniques, and methods of analyzing the data collected and determining causal links between the drugs, the patients, and the side effects.

Some disclosed methods comprise identifying a first population of people who have taken a first drug to treat a given disease and who have experienced a relatively high rate of occurrence of a first side effect as a result of taking the first drug, and identifying a second population of people who have taken a second drug to treat the given disease and who have experienced a relatively low rate of occurrence of the first side effect as a result of taking the second drug, wherein the first and second populations have generally homogenous personal characteristics. The method can further comprise determining a first biological target of the first drug, determining a second biological target of the second drug, determining a chemical feature that is present in the first drug and not present in the second drug, wherein the chemical feature is responsible for the first drug targeting the first biological target and not responsible for the second drug targeting the second biological target, and correlating the chemical feature and the first biological target with an increased likelihood of occurrence of the first side effect. In some embodiments, the method can further comprise treating a patient having the given disease with a drug that lacks the chemical feature to reduce the likelihood of occurrence of the first side effect.

Some disclosed methods comprise identifying a first population of people who have taken a first drug to treat a given disease and who have experienced a relatively high rate of occurrence of a first side effect as a result of taking the first drug, and identifying a second population of people who have taken the first drug to treat the given disease and who have experienced a relatively low rate of occurrence of the first side effect as a result of taking the first drug. The method can further comprise determining a biological target of the first drug, determining a personal characteristic that is relatively more common among the first population and relatively less common among the second population, and correlating the personal characteristic and the biological target with an increased likelihood of occurrence of the first side effect when taking the first drug. In some embodiments, the method can further comprise treating a patient having the given disease with the first drug based on a determination that the patient lacks the personal characteristic to reduce the likelihood of occurrence of the first side effect in the patient.

In any such methods, the method can also include collecting and using data from a variety of conventional and/or unconventional data sources that provide data regarding the intrinsic nature of drugs, data regarding known side effects of the drugs, and personal information about the people taking the drugs. Personal information about the people taking the drugs can comprise intrinsic information about the people, environmental information about the people, and/or behavioral information about the people. In some cases, personal information comprises information provided by the people or by other people on social media platforms. Additionally, population wide environmental and societal information can be incorporated.

In some methods, the biological target(s) are proteins. Some methods further comprise generating a drug-protein interaction network based on the drugs and the biological targets. The methods can further comprise generating a protein-protein interaction network based on the biological targets and the drug-protein interaction network. Some methods involve modifying a drug to remove a chemical feature linked to an undesired side effect and/or modifying a patient's behavior or environment to reduce the likelihood of the side effect occurring.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a flow chart illustrating an exemplary method described herein.

FIG. 2 is a flow chart illustrating another exemplary method described herein.

FIG. 3 is a flow chart illustrating yet another exemplary method described herein.

### DETAILED DESCRIPTION

Disclosed herein are methods and systems for discovering and analyzing drug-related side effects, which are also referred to herein as “off-target responses”. Side effects can be positive/beneficial side effects or negative/undesirable side effects. Further, the positive side effects can be utilized to repurpose a drug while undesirable side effects can be eliminated to make the drug(s) safer or to determine in which population the drug will be safest. Disclosed methods can utilize any one or more of a variety of data sources and data collection techniques to acquire data that can be utilized to identify side effects related to a particular drug and to determine that causal links between the drug, the patients, and the side effects.

More information related to the herein disclosed technology can be found in U.S. patent application Ser. No. 13/543,044, filed on Jul. 6, 2012, and entitled “SYSTEM AND METHOD FOR PERFORMING PHARMACOVIGILANCE”, U.S. patent application Ser. No. 13/549,890, filed on Jul. 16, 2012, and entitled “SYSTEM AND METHOD OF APPLYING STATE OF BEING TO HEALTH CARE DELIVERY”, and U.S. Provisional Patent Application No. 62/015,896, filed on Jun. 23, 2014, and entitled “TELEGENETICS”, the entire disclosures of which are hereby incorporated by reference in their entirety.

Exemplary sources of data related to side effects can include FDA side effects reports, drug and chemical databases, patient health records, reports regarding disease outbreaks (e.g., influenza), pollution records, weather reports, geographic and astronomical databases, patient specific behavioral records, social media sources both curated and in unstructured or raw form, global-population and ethnic-specific disease data banks, and many other sources. Any data source that may contain information relevant to particular drugs, patients taking the drugs, or the occurrence of side effects related to the drugs is an exemplary data source.

Disclosed methods can include an initial step of identifying side effects associated with a particular drug and a subsequent step of determining what causes each particular side effect when the patients are taking the particular drug. When more information about the side effects of a particular drug are known, more personalized and effective therapies can be developed for those patients taking the drug.

A particular drug can cause a particular side effect in some people taking the drug but not in others who are also taking the drug. For example, the statin-induced side effect of rhabdomyolysis occurs in about 1.5% of all people taking statins, but not in the others who are taking statins. Furthermore, across the statin class, Simvastatin causes rhabdomyolysis in more patients as compared to Pravastatin despite the fact that they both target the same enzyme, HMG-CoA Reductase. One question that follows is: why do the 1.5% of people experience a side effect of statins and not the others? Moreover, why do the patients experience the side effect with one statin but not with other? There can be many different kinds of answers to such questions. It may be that the 1.5% that experience the statin-induced side effect all have a common genetic trait that predisposes them to the side effect while the others do not have that genetic trait. Or it may be that the 1.5% who have the statin-induced side effect are taking another drug (e.g., macrolide antibiotic) that interacts with statins to cause rhabdomyolysis. These are very simplified examples, but in most cases the causal links between a drug and its side effects are more complicated. For example, it may be that there are several factors (e.g. food habits or preferences) that, when present, each increases the likelihood of a side effect occurring or work synergistically to cause a side effect.

Once a particular side effect is discovered and the causal link between a particular drug and the side effect is determined/identified, action can be taken to avoid the side effect (or in some cases encourage the side effect) in a patient. For example, in some cases the active pharmaceutical ingredient (API) in a pill can be chemically altered or changed to avoid a particular side effect while maintaining a therapeutic benefit of the active drug. For another example, environmental and/or behavior changes, such as a person's diet or other drug intake, can be adjusted to avoid the side effect. Furthermore, if more than one drug is known to provide the desired therapeutic benefit, but only one of the drugs causes a particular side effect in a particular patient or class of people to which the patient belongs, then the patient can be switched from the one drug that causes the side effect to one of the other drugs that does not cause the side effect while still obtaining the desired therapeutic result. In still other examples, a patient may experience a side effect at a geographical region when air pollution, pollen count, sun exposure, humidity, or other environmental factors contribute to the side effect. In such cases, the patient can move to a different location with different environmental conditions to reduce or eliminate the side effect.

### Data Types, Data Sources and Data Collection

One class of data sources are those sources that provide data regarding the intrinsic nature of a drug itself. These data sources can include drug and chemical databases that include information regarding the various compounds in the drug, their chemical structures, chemical properties, etc. These data sources may be provided by drug manufacturers, regulatory agencies, published literature, etc. Information about the drug itself can be useful in many different ways. For example, it may be discovered that there are several chemical variations of a particular class of drugs that each have similar therapeutic benefits, but different side effects. Or the different variations may interact differently with other drugs that patients may be taking or certain foods that patients may consume. Further, in some cases, the chemical structure of a drug may be altered in such a manner that an undesired side effect is eliminated for all people or for an entire class of people while maintaining the therapeutic benefit.

Another class of data sources are those sources that provide data regarding known side effects of a particular drug. These sources can include reports on trials conducted by the drug manufacturer, the FDA, independent research groups, or other regulatory bodies, which describe what side effects have been observed when the drug is widely prescribed or otherwise used by people. These data sources may also include data regarding the patients taking the drugs, both those that experienced the side effects and those that did not. These data can be used to detect other previously unreported or uncorrelated side effects that the same patients experienced. Further, side effects experienced from the use a particular drug can provide clues to what side effects may be caused by similar drugs.

Another class of data sources are those that provide personal information about a particular patient. These sources can include medical records, public records (e.g., DMV and other government records), social media accounts, etc. For more information regarding collecting and utilizing patient information from social media, see U.S. patent application Ser. No. 13/543,044. Data related to a particular patient can be grouped into various categories, such as intrinsic information, environmental information, and behavioral information (lifestyle, diet, exercise, relationship status, work type, etc.).

Intrinsic information can include genetic and epigenetic information, family history, anatomical information, physiological information, psychological and state of being information such as depression and mood status, current and past health conditions including current and past diseases and injuries, gender, age, height, weight, BMI, presence or absence of various anatomical features, previous surgeries or procedures, allergies, and many other types of information. For more information regarding collecting and utilizing psychological and state of being information, see U.S. patent application Ser. No. 13/549,890.

Environmental information can include a person's home location, work location, work setting (e.g., office vs. construction site), weather conditions (e.g., rain fall, humidity, temperature), natural conditions (e.g., pollen count, seasonal information), air pollution in the area or home and work, water pollution, contagious disease prevalence in area, proximity to other people having certain conditions (e.g., diseases), and other environmental information.

Behavioral information can include various lifestyle factors, diet, exercise types and patterns, smoking history, alcohol consumption, other drug use, sleep patterns, relationship status, educational background, relationship changes, work environment, changes in employment, changes in residence, membership in groups, religious/spiritual group affiliation, other social interactions, legal actions, etc.

Many of the data sources list above can provide more than one different type of data. For example, an FDA report may provide intrinsic information about a drug itself and may provide data about the drug's effectiveness and side effects found during clinical trials. As another example, social media sources may provide personal information about a particular person (e.g., that person may publically express personal information on Twitter or on PatientsLikeMe) and may provide information about additional side effects patients have experienced while taking a certain drug that were not initially discovered and reported when the drug was tested and provided to the patients.

It is generally desirable to utilize data from more verifiable/reliable sources than from less verifiable/reliable sources. Some sources, such as patient posts on PatientsLikeMe, may be less verifiable and less reliable, while other sources, such as FDA drug reports and historical weather charts, may be more verifiable and more reliable. Other sources, such as Wikipedia or private research reports, may have an intermediate level of reliability and verifiability. In cases of conflicting data or overlapping data, the most reliable and most verifiable data can be utilized and/or less reliable data can be corrected, verified to make it more reliable, or discarded.

Any combination of data sources can be used to collect data regarding drugs, people using the drugs, and side effects people experience while taking the drugs. The data may be found in many different forms. The data can be collected using various data acquisition techniques and stored in one or more databases or other data repositories. The disclosed methods can be used to collect more data, and a broader spectrum of data from a broader spectrum of source, than what is available from clinical drug trials and other standard testing routines. For example, a drug trial may test a drug on 1000 people and then collect information on the side effects those people experienced over a given period of time. However, the disclosed methods can incorporate data collected from many more people taking the drug, can collect a broader spectrum of data than what is collected during a drug trial, and can collect data over a longer period of time, all of which can lead to more accurate and useful results. For example, some side effects may not occur until after years of taking a drug, and drug trials that last for less than a year will not detect such side effects. Further, a drug trial that tests a drug on 1000 people is likely to miss a side effect that only occurs in 1-in-10,000 people taking the drug.

The collected data can be classified and organized by data type. Overlapping data can be deleted and conflicting or incorrect data can be corrected. The different data types can be integrated together into a synthesized data set that can be analyzed to identify side effects related to certain drugs and to determine causal links to the side effects.

### Data Analysis Methods

Based on the collected and synthesized data, various analytical processes can be carried out to achieve useful resulting information. Accumulated data may be processed or analyzed using various computing technologies, such as computer learning system, neural networks, cluster analysis, association rule approaches, etc. For example, analysis of the collected data may indicate that various drugs exist on the market that target the same ailment (e.g., “similar target medications”), but that they each result in a different prevalence of a certain side effect among a large group of patients. Using a particular example, different types of drugs for treating high cholesterol can result in different rates of chronic muscle ache in the patients taking those drugs. From this observation, in may be postulated that an unintentional, chemical compound difference among the various drugs, under certain circumstances, results in the variation in side effect manifestation. In other cases, it may be observed that the same exact drug has different side effects or different rates of a particular side effect among different classes of people that use the drug. From this type of observation, it may be postulated that there is one or more patient characteristics that cause a high likelihood of that particular side effect. Such postulations can then be tested and verified using data acquired from various data sources.

In some analysis methods, a decision tree can be used for interrogating drugs, patients, side effects, and their causal links. For example, an initial query may ask “Does every drug of a class of drugs (e.g., drugs for treating high cholesterol) cause the same particular side effect in one particular patient sub-population, but not in other patient sub-populations?” If the answer is yes, then the side effect can be considered intrinsic to that patient sub-population, and is likely causally related to one or more common characteristics among that sub-population. In this case, a following question can be “what are the most prevalent or common characteristics among that sub-population that are also less prevalent or uncommon among other patient sub-populations?” To answer this question, the data collected regarding the various patients can be analyzed using advanced techniques to identify the most likely correlation, and then those correlations can be investigated to determine chemical, biological, or other logical reasons that a certain personal characteristic would cause the particular side effect. For this, a personalized therapy approach can be developed for a particular sub-population known to have a set of genetic, epigenetic or other characteristics. For the particular sub-population, drugs can be selected that provide needed therapy but are least likely to cause undesired side effects.

If the answer to the initial question is that every drug of a class of drugs does not cause the same particular side effect X in one particular patient sub-population, then a following question can be “Do different drugs from the same class of drugs cause different side effects in the same patient population?” If the answer is yes, then it can be assumed that the cause of the side effects is tied to the inherent differences in the drugs themselves, and not differences among the patients. In this case, following inquiries can include “what are the differences in chemical structures of the various drugs in this class of drugs?” and “which of the differences can cause the observed side effect?” To answer these questions, data collected about the drugs can be analyzed, such as data acquired from drug and chemical compound databases. Once chemical differences are identified, each difference can be investigated to possible causal links to the observed side effects.

In some methods, cluster-based feature identification techniques can be used. In an exemplary method, a drug exhibiting a particular side effect can be initially selected. The patient population taking the drug (“POP”) can then be partitioned into a set of those patients who are exhibiting the side effect (“EXHIBIT”) and a set of those patients who are not exhibiting the side effect (“NONE”). The EXHIBIT set and the NONE set can then be analyzed using cluster analysis, both individually and collectively as the whole set POP. Each set can be clustered, and for each cluster, a set of key features or a representative central element (e.g., a centroid) for the cluster can be determined. For example, a centroid for the NONE cluster (“Cent-N”), a centroid for the EXHIBIT cluster (“Cent-E”), and a centroid for the POP cluster (“Cent-P”) can be determined. The strengths of each feature or centroid can then be differentiated. For example, the method can include determining what is dominant in Cent-N but not in Cent-P, what is dominant in Cent-E but not in Cent-P, and what is dominant in Cent-E, but not in Cent-N. The method can also include correlating the key differences among the clusters with a potential effect. For example, if we assume Cent-E is found to have a key feature (e.g., hypertension) Cent-P is found to have only limited strength for that feature, and Cent-N is found to have little or no strength for that feature, then a postulation can be formed that the particular side effect for the particular drug is in people who have hypertension. The key difference between the EXHIBIT set and the NONE set might not be drug related, but still may have medical implications (e.g., lack of exercise, poor diet, too much work).

In some methods, association rule based feature identification techniques can be used. In an exemplary method, the entire population taking a particular drug POP can be studied for as many features as are known about those patients, and from this analysis one or more association rules can be determined. For example, it may be determined that patients having both genetic susceptibility of a drug interacting enzyme (feature 1) and variability in a drug metabolizing mechanism (feature 2) are more likely to exhibit a particular side effect when taking the particular drug. More than one such association rules can be determined, and for each rule a level of confidence and support can be determined, which indicates how strong the association rule is at predicting who may or may not have the side effect when taking the drug. Based on the determined association rules and the associated confidence and support levels, it can be postulated that a particular side effect for a particular drug is likely to occur in people who have the identified features of the association rules with sufficiently high confidence and support. Again, the key difference might not be drug related, but still with medical implications (e.g., lack of exercise, poor diet, too much work).

Once a likely causal link for a particular drug and a particular side effect is determined, verification of the postulated causal link can be conducted. The verification process can again utilize data collected for a wide variety of sources, including non-traditional sources, and can utilize newly collected data that are targeted specifically at verifying the postulated causal link.

In some methods, the verification process can include verifying that a chemical structure difference (“CHEM-DIF”) among different drugs of a class of drugs is a causal link to a particular side effect. Such verification methods can include correlating postulated features (related to the drug or to the patients) in coordination with CHEM-DIF as possible inducers of the particular side effect. The method can include determining if one or more of the determined features catalyze the CHEM-DIFF so as to explain why a particular population segment is more commonly affected by the CHEM-DIF to yield the particular side effect. Using the example of various different drugs for treating high cholesterol, the method can correlate the feature of patient intake of grapefruit juice with the CHEM-DIF among the high cholesterol drugs as being a causal link to the side effect of chronic muscle aches. In this example, a certain compound in grapefruit juice causes certain high cholesterol drugs to bind with the incorrect biological target in a patient, which leads to muscle aches. After such a correlation is determined, the causal mechanism that actually causes the side effect can be studied and verified. With the causal mechanism identified, changes in the chemical structure of the drugs can be made to eliminate or lessen the side effect, or changes in the patient's behavior (e.g., reduce grapefruit juice consumption) can be suggested to eliminate or lessen the side effect.

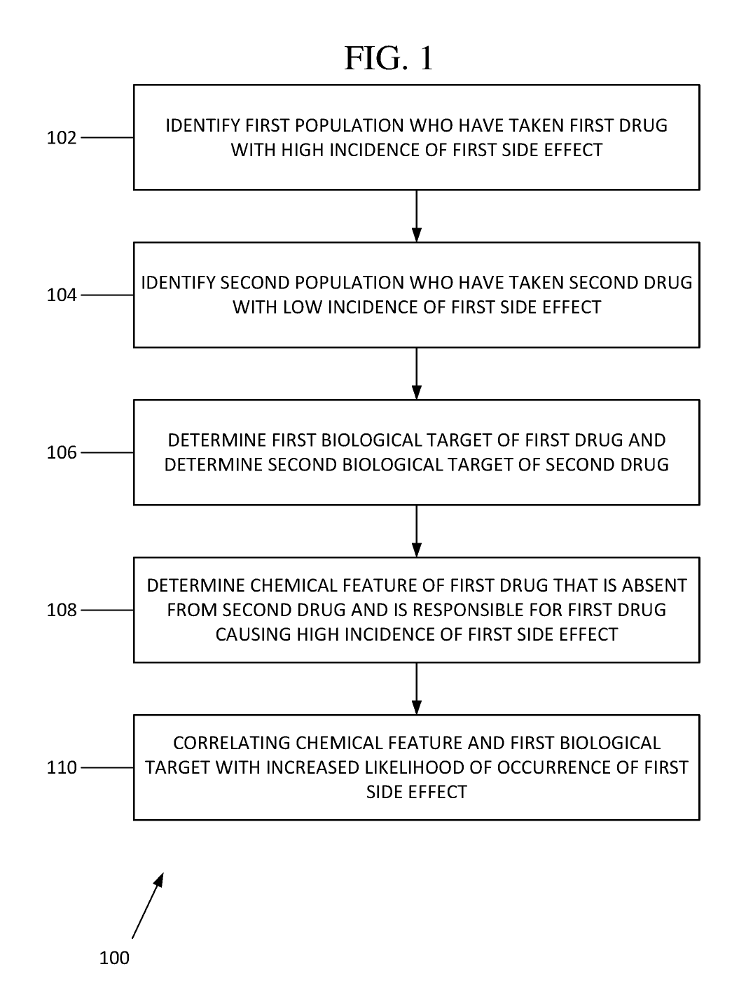

FIG. 1 is a flow chart illustrating an exemplary method 100 for analyzing drug-related side effects where an intrinsic difference between different drugs for treating a common disease is correlated with the occurrence of a particular side effect. At 102, a first population is identified who have taken a first drug and exhibited a relatively high incidence rate for a first side effect of the first drug. At 104, a second population is identified who have taken a second drug and exhibited a relatively low incidence of the first side effect. Here, the first and second populations are from a common population and have homogeneous personal characteristics, and the first and second drugs are different. At 106, first and second biological targets are determined from the first and second drugs, respectively. At 108, the method includes determining a chemical feature that is present in the first drug and not present in the second drug, wherein the chemical feature is responsible for the first drug causing the relatively higher rate of occurrence of the first side effect compared to the second drug that lacks the chemical feature. At 110, the method includes correlating the chemical feature and the first biological target with an increased likelihood of occurrence of the first side effect. The method may further include additional elements, such as treating a patient having the given disease with a drug that lacks the chemical feature to reduce the likelihood of occurrence of the first side effect.

The following is an example of the exemplary method 100 illustrated in FIG. 1 with respected to two antihistamine drugs: diphenhydramine and fexofenadine. A first population of older adults is identified (102). The first population have taken a first drug diphenhydramine and have had a high incidence of a first side effect of incidence of cognitive impairment. A second population of older adults is identified (104). The second population have taken a second drug fexofenadine and have had a low incidence of the first side effect of cognitive impairment. A first biological target of the first drug diphenhydramine is determined and a second biological target of the second drug fexofenadine is determined (106). The chemical feature of the first drug diphenhydramine that is absent from the second drug fexofenadine and is responsible for the high incidence of the first side effect of cognitive impairment is determined (108). It is determined that the differences appear to lie within chemical moiety responsible for anti-cholinergic functions. The chemical feature and the first biological target are correlated with the increased likelihood of occurrence of the first side effect (110). The method may further include additional elements, such as treating a patient having the given disease with the second drug fexofenadine that lacks the chemical feature to reduce the likelihood of occurrence of the first side effect of cognitive impairment. Additionally and/or alternatively, the method may also include additional elements, such as modifying the first drug diphenhydramine to alter the molecular structure of the drug, thereby removing the chemical feature that may cause an occurrence of the first side effect of cognitive impairment in the patient, while maintaining the therapeutic effect of the first drug.

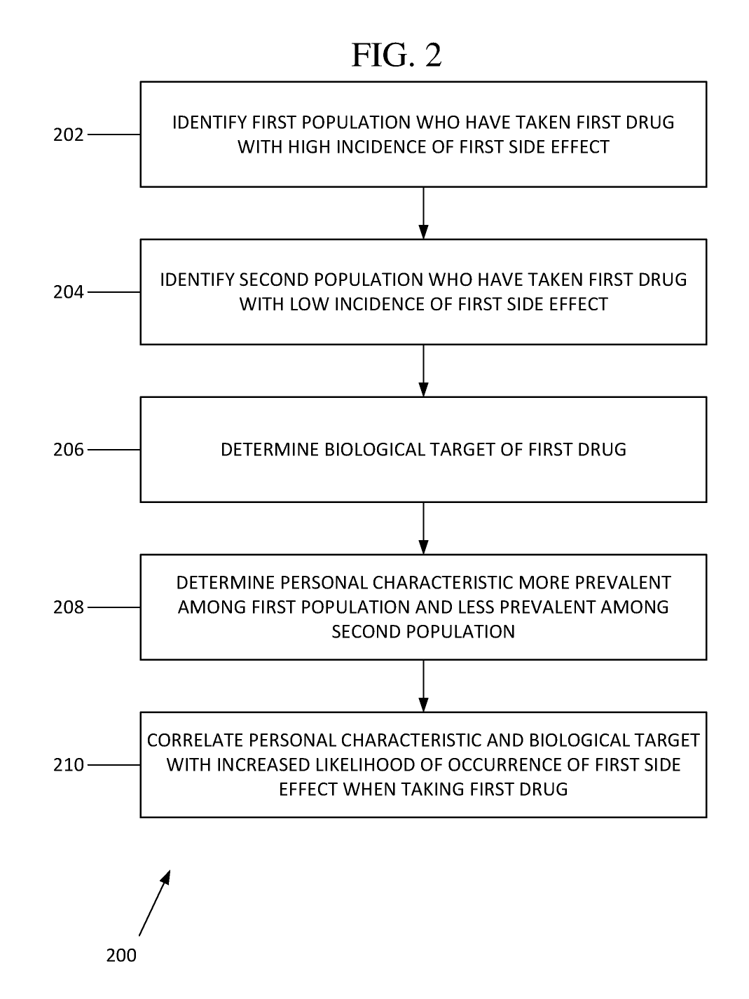

FIG. 2 is a flow chart illustrating another exemplary method 200 for analyzing drug-related side effects where a personal characteristic difference between different patients taking a common drug is correlated with the occurrence of a particular side effect. At 202, the method comprises identifying a first population of people who have taken a first drug to treat a given disease and who have experienced a relatively high rate of occurrence of a first side effect as a result of taking the first drug. At 204, the method comprises identifying a second population of people who have taken the same first drug to treat the given disease and who have experienced a relatively low rate of occurrence of the first side effect as a result of taking the first drug. Here, the first and second populations are investigated to determine key differences in personal characteristics that are associated with causing the side effect. At 206, the method comprises determining a biological target of the first drug. At 208, the method comprises determining a personal characteristic that is relatively more common among the first population and relatively less common among the second population. At 210, the method comprises correlating the personal characteristic and the biological target with an increased likelihood of occurrence of the first side effect when taking the first drug. The method may also include additional elements, such as treating a patient who has the given disease, has the personal characteristic, and is taking the first drug, by modifying the patient's behavior and/or environment to eliminate or reduce the personal characteristic to reduce the likelihood of occurrence of the first side effect in the patient. Additionally and/or alternatively, the method may also include additional elements, such as treating a patient who has the given disease, has the personal characteristic, and is taking the first drug, by providing a treatment plan that may include modification of the patient's behavior and/or environment to eliminate or reduce the personal characteristic to reduce the likelihood of occurrence of the first side effect in the patient.

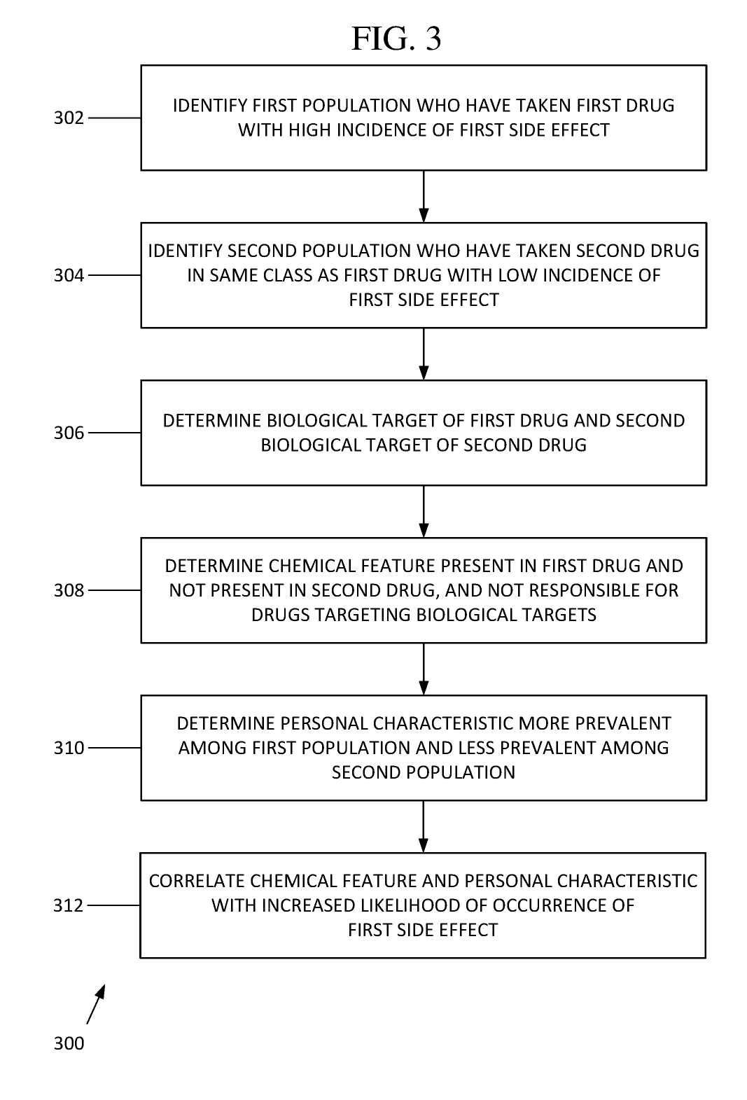

FIG. 3 is a flow chart illustrating another exemplary method 300 for analyzing drug-related side effects where a personal characteristic difference between different patients taking two different drugs of the same class of drugs is correlated with the occurrence of a particular side effect. At 302, the method comprises identifying a first population of people who have taken a first drug to treat a given disease and who have experienced a relatively high rate of occurrence of a first side effect as a result of taking the first drug. At 304, the method comprises identifying a second population of people who have taken a second drug of the same class of drugs to treat the given disease and who have experienced a relatively low rate of occurrence of the first side effect as a result of taking the first drug. Here, the chemical differences between the first and second drugs are investigated and the first and second populations are investigated to determine differences in personal characteristics that are associated with causing the side effect. At 306, the method comprises determining biological targets of the first and second drugs. At 308, the method comprises determining a chemical feature present in the first drug and not present in the second drug, where the chemical feature is not responsible for the two drugs targeting their respective biological targets. For example, the first drug may include an added compound included for a purpose other than interacting with the first biological target. At 310, the method comprises determining a personal characteristic that is relatively more prevalent among the first population and relatively less prevalent among the second population. At 312, the method comprises correlating the chemical feature and the personal characteristic with an increased likelihood of occurrence of the first side effect. The method may also include additional elements, such as treating a patient having the given disease with the a drug lacking the chemical feature based on a determination that the patient has the personal characteristic to reduce the likelihood of occurrence of the first side effect in the patient. Additionally and/or alternatively, the method may also include additional elements, such as modifying a drug to alter the molecular structure of the drug, thereby removing the chemical feature that may cause an occurrence of the first side effect in the patient. Additionally and/or alternatively, the method may also include additional elements, such as treating a patient having the given disease with the a drug that is modified to remove the chemical feature based on a determination that the patient has the personal characteristic to reduce the likelihood of occurrence of the first side effect in the patient.

### Systems-Level Interactome Analysis

In a reductionist approach for understanding biological phenomenon, macromolecules such as proteins are visualized in linear pathways where external cues are translated into biological signals in a sequential manner. However, discovery biological processes at molecular and atomic levels have revealed inter-connection of pathways to form a network. Furthermore, networks are then inter-connected to form large interactome where many networks connections diversify signals into a multitude of directions to generate systems level complexity. Thus, a critical step towards unraveling the complex molecular relationships in living systems is to map protein-to-protein interactions. Achieving a map of protein-protein interactions within a living system can allow the construction of the interaction network of the system and the identification of the corresponding central nodes that can be critical for a function, together with homeostasis, and genomic/proteomic alterations and metabolic activities of human physiology at the system level. Data on the human interactome are particularly relevant for current biomedical research because the location of the proteins in the interactome network can allow the evaluation of their centrality and can redefine of the potential value of such protein as a drug target. Network visualization of drug-target, target-disease and disease-gene associations can provide helpful information for discovery of new therapeutic indications and/or adverse effects of old drugs.

Once protein targets are identified drug-protein interaction networks can be generated. Tools can be used to create/identify such networks and understand the pathways involved. For example, IPA® from Ingenuity® Systems is a web based software application that helps understand complex “omics” data at multiple levels by integrating data from a variety of experimental platforms and providing insight into the molecular and chemical interactions, cellular phenotypes, and disease processes of the studied system. IPA® and similar tools can provide insight into the causes of observed gene expression changes and into the predicted downstream biological effects of those changes.

Databases of known interaction networks can also be utilized as a data source in the disclosed methods. For example, STRING is a database of known and predicted protein interactions derived from four different sources, and thus quantitatively integrate interaction data and transfers information between the organisms where applicable. Another tool, Cytoscape is an open source software platform for visualizing complex networks and integrating with gene expression profiles and other state data and can be used to visualize and analyze network graphs of any kind involving nodes and edges.

The protein targets identified through data scouring can be used to create networks and map existing pathways into the networks. This can be used to create a protein signaling network or gene expression network connecting the protein targets Existing tools, such as the IPA® tool, can be used for generating such networks.

In exemplary methods, the identified drug/target interactions can be loaded into Cytoscape to create protein-protein interaction networks or drug target networks. The existing interactions can be overlapped into a protein-protein interaction database to identify signaling pathways involved with the protein targets. Various similarity measures such as structural similarity, chemical similarity, genomics similarity, etc., combined with machine learning, data mining, and data analytical, including graphical tools can be used to build and visualize networks. Graph database queries, such as those commonly supported in NO-SQL database engines, can be used to further interrogate the network.

To predict unknown interactions, a network map constructed with known interactions and similarity measures from the protein targets extracted can be used. Several algorithms derived from complex network theories, such as drug-based similarity inference (DBSI), target-based similarity inference (TBSI), and network-based inference (NBI), can be used for construction of a predictive biomathematical model for unknown interactions.

IPA® leverages the Ingenuity Knowledge Base, a repository of biological interactions and functional annotations created from millions of individually modeled relationships between proteins, RNAs, genes, isoforms, metabolites, complexes, cells, tissues, drugs, and diseases. These modeled relationships, or Findings, include rich contextual details and link to the original sources of the information. Findings are manually curated and reviewed for accuracy and detail, and follow strict quality control processes. The Ingenuity Knowledge Base provides a reliable resource for searching relevant and substantiated knowledge from the various sources, and for interpreting experimental results in the context of larger biological systems.

Ingenuity® structures all of the biological and chemical content in the Ingenuity Knowledge Base using the Ingenuity Ontology. The structured content enables computation and inferencing, ensures semantic and linguistic consistency, and supports the integration and mapping of content from multiple sources. In addition, the curation process can include relevant contextual details about the relationships, such as species specificity, cell type/tissue context, type of mutations, direction of change, post-translational modification sites, epigenetic modifications, and/or experimental methods used. These network identification/creation techniques and curation processes can be used to identify relationships that correlate or associate one or more particular drug compounds to diseases, phenotypes and/or toxic/adverse effects.

Some methods can further comprise interrogating the drug-protein and protein-protein interaction networks via graphical database tools. Some methods can further comprise storing the result of the interrogation of the drug-protein and protein-protein interaction networks via graphical database tools, such as in a cloud service supporting the communal sharing of and/or commenting on the results. Some methods can further comprise supporting social media postings on the commenting of the results.

### General Considerations

For purposes of this description, certain aspects, advantages, and novel features of the embodiments of this disclosure are described herein. The disclosed methods, apparatuses, and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present or problems be solved.

Integers, characteristics, qualities, and other features described in conjunction with a particular aspect, embodiment, or example of the disclosed technology are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods.

As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.” As used herein, the term “coupled” generally means linked mechanically, electrically, chemically, and/or linked via any wireless or wired data transmission technology, and does not exclude the presence of intermediate elements between the coupled items absent specific contrary language.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following exemplary claims. We therefore claim all that comes within the scope of these claims.

1. A method comprising:
identifying a first population of people who have taken a first drug to treat a given disease and who have experienced a relatively high rate of occurrence of a first side effect as a result of taking the first drug;
identifying a second population of people who have taken a second drug to treat the given disease and who have experienced a relatively low rate of occurrence of the first side effect as a result of taking the second drug;
determining a first biological target of the first drug and a second biological target of the second drug, the first and second biological targets being associated with the given disease;
determining a chemical feature that is present in the first drug and not present in the second drug, wherein the chemical feature is responsible for the first drug having the relatively high rate of occurrence of the first side effect;
correlating the chemical feature of the first drug with an increased likelihood of occurrence of the first side effect; and
treating a patient having the given disease with a drug that lacks the chemical feature of the first drug.

2. The method of claim 1, wherein the method comprises determining a biological mechanism that causally relates the chemical feature, the first drug and the first side effect.

3. The method of claim 1, wherein the first and second biological targets are proteins and the method further comprises generating a drug-protein interaction network based on the first and second drugs and the first and second biological targets.

4. The method of claim 3, wherein the method further comprises generating a protein-protein interaction network based on the first and second biological targets and the drug-protein interaction network.

5. The method of claim 1, wherein treating a patient having the given disease with a drug that lacks the chemical feature comprises modifying a drug to remove the chemical feature and then treating the patient with the modified drug.

6. The method of claim 1, wherein the first population and the second population have homogeneous personal characteristics or are the same population.

7. The method of claim 1, further comprising:
determining the first population and the second population from a general population of people who took medication to treat the given disease using data collected from data sources that provide data regarding the intrinsic nature of first and second drugs, from data sources that provide data regarding known side effects of the first and second drugs, and from data sources that provide personal information about the general population of people.

8. The method of claim 7, wherein the data from data sources that provide personal information about the general population of people comprises intrinsic information about the general population of people, environmental information about the general population of people, and behavioral information about the general population of people.

9. The method of claim 8, wherein intrinsic information about the general population of people comprises genetic and epigenetic information.

10. The method of claim 8, wherein the data from data sources that provide personal information about the general population of people comprises information provided by the general population of people on social media platforms.

11. A method comprising:
identifying a first population of people who have taken a first drug to treat a given disease and who have experienced a relatively high rate of occurrence of a first side effect as a result of taking the first drug;
identifying a second population of people who have taken the first drug to treat the given disease and who have experienced a relatively low rate of occurrence of the first side effect as a result of taking the first drug;
determining a biological target of the first drug;
determining differences in personal characteristics of the first population and the second population;
correlating the differences in personal characteristics with an increased likelihood of occurrence of the first side effect when taking the first drug; and
treating a patient having the given disease, wherein treating the patient is based on a determination that the patient has personal characteristics that are correlated with the first drug and the first side effect, and further wherein treating the patient includes one or both of chemically altering the first drug to avoid the first side effect while maintaining a therapeutic benefit of the first drug and treating the patient with the second drug.

12. The method of claim 11, wherein the method comprises determining a biological mechanism that causally relates the personal characteristic, the biological target, and the first side effect.

13. The method of claim 11, wherein the biological target is a protein and the method further comprises generating a drug-protein interaction network based on the first drug and the biological target.

14. The method of claim 13, wherein the method further comprises generating a protein-protein interaction network based on the biological target and the drug-protein interaction network.

15. The method of claim 11, wherein treating a patient having the given disease comprises modifying the patients behavior or environment such that the patient lacks the personal characteristics that are correlated with the first drug and the first side effect.

16. The method of claim 11, further comprising:
determining the first population, the second population and the personal characteristic using data collected from data sources that provide data regarding the intrinsic nature of first drug, from data sources that provide data regarding known side effects of the first drug, and from data sources that provide personal information about the first and second populations.

17. The method of claim 16, wherein the data from data sources that provide personal information comprises intrinsic information about the first and second populations, environmental information about the first and second populations, and behavioral information about the first and second populations.

18. The method of claim 17, wherein the data from data sources that provide personal information about the first and second populations comprises information provided by the first and second populations on social media platforms.

19. A method comprising:
identifying a first population of people who have taken a first drug to treat a given disease and who have experienced a relatively high rate of occurrence of a first side effect as a result of taking the first drug;
identifying a second population of people who have taken a second drug to treat the given disease and who have experienced a relatively low rate of occurrence of the first side effect as a result of taking the second drug;
wherein the first and second drugs are different but are from a same class of drugs for treating the given disease;
determining a first biological target of the first drug and a second biological target of the second drug, the first and second biological targets being associated with the given disease;
determining a chemical feature that is present in the first drug and not present in the second drug, wherein the chemical feature is not responsible for the first and second drugs targeting the first and second biological targets;
determining a personal characteristic that is relatively more prevalent among the first population and relatively less prevalent among the second population;
correlating the chemical feature and the personal characteristic with an increased likelihood of occurrence of the first side effect; and
treating a patient having the given disease and having the personal characteristic with a drug that lacks the chemical feature.

20. The method of claim 19, wherein the method comprises determining a biological mechanism that causally relates the chemical feature, the first biological target, the personal characteristic, and the first side effect.

21. The method of claim 19, wherein the first and second biological targets are proteins and the method further comprises:
generating a drug-protein interaction network based on the first and second drugs and the first and second biological targets; and
generating a protein-protein interaction network based on the first and second biological targets and the drug-protein interaction network.

22. The method of claim 19, further comprising:
determining the first population, the second population, the chemical difference, and the personal characteristic using data collected from data sources that provide data regarding the intrinsic nature of first drug, from data sources that provide data regarding known side effects of the first drug, and from data sources that provide personal information about the first and second populations;
wherein the data from data sources that provide personal information comprises intrinsic information about the first and second populations, environmental information about the first and second populations, and behavioral information about the first and second populations.

23. The method of claim 19, wherein treating the patient further comprises modifying the drug to remove the chemical feature while maintaining a therapeutic benefit of the drug.

24. A system comprising computing hardware configured to perform the method of claim 1.

25. A computer readable storage device comprising instructions for causing one or more computing devices to perform the method of claim 1.
